(19) United States
(12) Patent Application Publication
(10) Pub. No.: US 2015/0231212 A1
(43) Pub. Date: Aug. 20, 2015

(54) ST6GAL-1 MEDIATED MODULATION OF HEMATOPOIESIS

(71) Applicant: Health Research, Inc., Buffalo, NY (US)

(72) Inventors: Joseph Lau, Hamburg, NY (US); Mehrab Nasiri Kenari, Amherst, NY (US)

(21) Appl. No.: 14/703,210

(22) Filed: May 4, 2015

Related U.S. Application Data
(63) Continuation-in-part of application No. 14/128,642, filed on Apr. 8, 2014.
(60) Provisional application No. 61/987,676, filed on May 2, 2014.
(60) Provisional application No. 61/501,093, filed on Jun. 24, 2011.

(51) Int. Cl. A61K 38/45; A61K 49/00

(52) U.S. Cl. CPC A61K 38/45; A61K 49/0008

(57) ABSTRACT

Provided are methods for reducing inflammation and allergic reactions and for reducing the severity of autoimmune disorders. The method involve administering to an individual a composition that contains recombinant α2,6-sialyltransferase (ST6Gal-1). The disclosure also provides a pharmaceutical preparation that contains recombinant ST6Gal-1 and which is suitable for administration to an individual to reduce inflammation or allergic reactions, including acute allergic reactions. A method for identifying agents capable of reducing acute allergic reactions is also provided

### CROSS-REFERENCE TO RELATED APPLICATIONS

This application claims priority to U.S. provisional application No. 61/987,676, filed May 2, 2014, and this application is a continuation in part of U.S. patent application Ser. No. 14/128,642, filed Apr. 8, 2014, which is the National Phase of International application no. PCT/US2012/043966, filed Jun. 25, 2012, which claims priority to U.S. provisional application No. 61/501,093, filed Jun. 24, 2011, the disclosures of each of which are incorporated herein by reference.

### GOVERNMENT FUNDING

This invention was made with government support under grant no. numbers AI-056082 and HL-078429 awarded by the National Institutes of Health. The government has certain rights in the invention.

### FIELD

The present disclosure relates generally to modulating immune responses and more specifically to methods for prophylaxis and/or therapy of conditions correlated with inflammation and allergic reactions.

### BACKGROUND

Hematopoiesis is the mechanism that produces circulating blood cells and certain other cells that participate in immune responses in various tissues. Inflammation is part of a complex biological response to harmful stimuli, such as pathogens, damaged cells, irritants or tissue malfunction. Inflammation is normally a protective attempt to remove the injurious stimuli and to initiate the healing process. However, dysregulated inflammation or failure to resolve inflammation is deleterious. Many disease conditions that affect vast numbers of people involve aberrant activity of such cells, including various cancers, autoimmune disorders, organ and tissue transplantation rejections, and multiple conditions that involve undesirable inflammation as a component of disease etiology. Additionally, the prevalence of allergic reactions in the human population has been steadily arising, particularly acute allergic reactions that involve risk of or manifestation of anaphylaxis in response to allergens such as venom and components of certain foods, such as peanuts, tree nuts, shellfish, and others. Thus there is an ongoing and unmet need for new approaches to prophylaxis and therapy for disorders that involve undesirable inflammation or allergic reactions, particularly acute allergic reactions which can be life threatening. The present disclosure addresses these and other needs.

### SUMMARY

The present disclosure comprises administering to an individual in need thereof a composition comprising recombinant α2,6-sialyltransferase (ST6GAL-1), wherein the administration results in a reduction of one or more symptoms of inflammation or an allergic reaction or an autoimmune condition. The method is thus broadly applicable to prophylaxis and/or therapy of any condition that is positively correlated with inflammation, or allergic reactions, as well as a variety of other conditions that involve the activity of blood cells, such as autoimmune disorders. Also provided is a method for identifying agents that are candidates for use in prophylaxis and/or therapy of allergic reactions.

### BRIEF DESCRIPTION OF THE FIGURES

FIG. 1 provides a graphical representation of data showing ST6Gal-1 profiles of animals undergoing OVA or ABPA models of allergic pulmonary inflammation.

FIG. 2 provides a graphical representation of data showing greater neutrophilia in Siat1ΔP1 and Siat1-null animals.

FIG. 3 provides a graphical representation of data showing suppression of G-CSF elicited release of white cells, including neutrophils, by recombinant ST6GAL-1 infusion i.v.

FIG. 4 provides a graphical representation of data showing LSK (Lin−:Sca+:cKit+) cells from Siat1ΔP1 mice have greater proliferation in vitro.

FIG. 5 provides a graphical representation of data showing supplementation of in vitro cultures of C57BL/6 bone marrow cells with recombinant ST6GAL-1 (rST6G) resulted in depressed IL-3/G-CSF dependent and IL-5 dependent colonies.

FIG. 6 provides a graphical representation of data showing Siat1ΔP1 donors and recipients are less able to retain transfused donor stem cells.

FIG. 7 provides a graphical representation of data showing elevated eosinophil infiltration in the bronchoalveolar lavage of ST6Gal-1-deficient mice upon allergen provocation.

FIG. 8 provides a graphical representation of data showing allergic airway inflammation is attenuated by bolstering systemic ST6Gal-1.

FIG. 9 provides a graphical representation of data showing loss of 33% of total nucleated cell numbers in bone marrow after administration of recombinant ST6GAL-1.

FIG. 10 provides a graphical representation of data showing flow analysis of bone marrow nucleated cells for CD11b and Ly6G showing depletion of granulocyte reservoir in marrow (circled population) after administration of recombinant ST6Gal-1.

FIG. 11 provides a graphical representation of data showing a decrease in total white cell counts (WBC), lymphocytes (LYMPH), and platelets (PLT) in circulation after administration of recombinant ST6Gal-1.

FIG. 12 provides a graphical representation of data showing that transient depression of circulatory ST6Gal-1 accompanies acute airway inflammation.

FIGS. 13A, 13B and 13C provide summaries of data showing greater neutrophilic acute airway in animals with depressed circulatory ST6Gal-1 levels.

FIGS. 14A, 14B and 14C provide graphical summaries of data showing that intravenous rST6G administration depresses myelopoiesis and alters inflammatory cell availability.

FIGS. 15A, 15B, 15C and 15D provide graphical and photographic representations of data showing mitigation of neutrophilic infiltration in an acute airway by recombinant ST6Gal-1.

FIG. 16 provides a graphical representation of data showing that r ST6Gal-1 suppressed inflammatory cytokine release during acute airway inflammation.

FIGS. 17A, 17B and 17C provide representations of data showing inflammatory cytokine release by macrophage was attenuated by ST6Gal-1.

FIG. 18 provides a graphical representation of data showing St6gal1-dP1 marrow, at baseline, has greater eosinophil colony-forming activity than wild-type C57BL/6 marrow. C57BL/6 (open bar; N=3) and St6gal1-dP1 (hatched bar; N=3) marrow cells were place in semisolid media (Methocult 3234, Stem CellTechnologies) supplemented with 25 ng/ml mouse rIL-5. Colonies with at least 50 cells were counted at Day 7. Shown are CFU/10e5 starting marrow cells.

### DETAILED DESCRIPTION

The present disclosure provides in various embodiments compositions and methods treating inflammation, or allergic reaction, or autoimmune disorders, in a subject in need thereof.

The method comprises administering to the individual a composition comprising an effective amount of recombinant α2,6-sialyltransferase (ST6Gal-1). In embodiments, the administration is for treating d inflammation, and/or allergic reactions, and/or autoimmune disorders.

The terms “treat,” “treating” or “treatment” as used herein, include alleviating, abating or ameliorating a disease or condition symptoms, preventing additional symptoms, ameliorating or preventing the underlying causes of symptoms, inhibiting the disease or condition, e.g., arresting the development of the disease or condition, relieving the disease or condition, causing regression of the disease or condition, relieving a condition caused by the disease or condition, or stopping the symptoms of the disease or condition. The terms “treat,” “treating” or “treatment” include, but are not necessarily limited to, prophylactic and/or therapeutic treatments.

In embodiments the disclosure encompasses administering an effective amount of ST6Gal-1. The terms “effective amount” as used herein refers to a sufficient amount of ST6Gal-1 which will relieve to some extent one or more of the symptoms of the disease or condition being treated. The result can be reduction and/or alleviation of the signs, symptoms, or causes of a disease.

The data presented herein demonstrate that infusion of recombinant soluble ST6Gal-1 results in (a) suppression of new inflammatory cell production; (b) suppression of release of inflammatory mediators; and (c) promotion of release of anti-inflammatory mediators. Some of the data presented herein are generated in response to challenge of mice with an infectious agent. Thus, the methods provided by this disclosure are expected to be useful for treating conditions wherein management of inflammatory conditions is desirable. Such conditions include but are not limited to acute and chronic inflammatory condition such as inflammation of the airway, sepsis, Crohn's disease, and cardiovascular diseases. In an embodiment, the inflammation is caused by exposure to foreign substance or tissue stress and malfunction. In embodiments, the foreign substance can be cancer cells, or an infectious agent such as a bacteria, virus, mold, or foreign cells or substances released by an infectious agent, or homeostatic imbalance of one or several physiological systems (diabetes, obesity, cardiovascular diseases). The invention also provides for treating chronic inflammatory diseases, which include but are not necessarily limited to chronic obstructive pulmonary disease (COPD), irritable bowel syndrome, and atherosclerosis.

The invention also provides for treating conditions that are characterized by allergic reactions. Thus, in various embodiments, the invention is useful for inhibiting or lessening the severity of, for instance, Type I hypersensitivity reactions and/or late phase allergic responses. Non-limiting examples of allergic reactions for which the present invention can provide a prophylactic and/or therapeutic benefit include allergic rhinitis, food allergies, asthma and related airway inflammatory conditions, allergic reactions caused by envenomation or medications.

In connection with prophylaxis and/or therapy of Type I hypersensitivity reactions, those skilled in the art will recognize that they are allergic reactions in response to an antigen, wherein the antigen is an allergen. Those skilled in the art will also recognize that Type I hypersensitivity reactions also apply to conditions of anaphylaxis of idiopathic origins. In embodiments, the allergic reaction that is reduced according to the disclosure is an allergic reaction that is stimulated upon re-exposure to an allergen and also idiopathic anaphylaxis where pre-exposure to an allergen cannot be clearly identified. Without intending to be bound by any particular theory, the present disclosure thus comprises in various, non-limiting embodiments, inhibiting sensitization of mast cells or basophils, and/or inhibiting secretion of compounds such as histamine, leukotrienes, prostaglandins, cytokines, and combinations thereof. In embodiments, the disclosure includes inhibiting vasodilation and/or smooth-muscle contraction that is typical of type I hypersensitivity reactions. In embodiments, the disclosure provides for preventing or inhibiting an immediate allergic reaction, and/or a late-phase allergic reaction. It is considered that the immediate hypersensitivity reaction arises within minutes after exposure to an allergen; the late-phase reaction takes place within 2-4 hours after exposure and is characterized at least in part by the release of cytokines from several types of immune cells. In embodiments, the disclosure includes inhibiting production of compounds selected from IL-2, IL-6, IL-10, IL1β, TNFα, histamine, and combinations thereof. In one embodiment, the disclosure provides for preventing, inhibiting, or reversing anaphylaxis, including but not necessarily limited to anaphylactic shock.

The invention also provides for prophylaxis and/or therapy of autoimmune diseases characterized by an inappropriate immune response against self-antigens or other substances that are normally present in the body. Non-limiting examples of autoimmune diseases for which the present invention can provide a prophylactic and/or therapeutic benefit include those which are characterized by type II, III or IV hypersensitivity. Particular, non-limiting examples include celiac disease, Crohn's disease, diabetes mellitus type 1, eosinophilic fasciitis, eosinophilic gastroenteritis, gastritis, Graves' disease, hypogammaglobulinemia, idiopathic inflammatory demyelinating diseases, thrombocytopenic purpura, rheumatoid arthritis, lupus erythematosus, myasthenia gravis, pernicious anaemia, psoriasis, Sjögren's syndrome, and ulcerative colitis.

The disclosure is also suited for use in connection with transplantations, such as allogeneic and autologous bone marrow transplantations, stem cell transplantation, adoptive T cell therapies, and tissue and organ transplantations, such as for prophylaxis and/or therapy of transplant rejection processes, including but not limited to graft versus host disease. Thus, in various embodiments, the individual to whom a composition comprising recombinant ST6Gal-1 is administered according to the invention is a candidate for, or is a recipient of a cell, tissue or organ transplantation.

The invention disclosure also provides a pharmaceutical preparation suitable for administration to an individual to reduce inflammation and/or an allergic reaction in the individual. The pharmaceutical preparation comprises recombinant ST6Gal-1 and a pharmaceutically acceptable carrier. In embodiments the pharmaceutical composition can also comprise an antibiotic, or an anti-allergic drug. In embodiments, the pharmaceutical composition is a liquid formulation. In embodiments, the liquid formulation is for use in intravenous administration, or is for use as an inhalant.

In embodiments the disclosure further provides products, e.g. articles of manufacture, or kits, which comprise ST6GAL-1 pharmaceutical preparations. The products comprise isolated and/or purified ST6GAL-1, and packaging and/or printed material. In one embodiment, the instant disclosure includes a closed or sealed package that contains an ST6GAL-1 pharmaceutical preparation. In certain embodiments, the package can comprise one or more closed or sealed vials, bottles, blister (bubble) packs, or any other suitable packaging for the sale, or distribution, or use of the ST6GAL-1 pharmaceutical agents. In embodiments, the product includes a syringe. For example, the syringe may be a tuberculin syringe wherein a small volume is needed, such as 0.1, 0.2, 0.9, up to 1.0 ml. The kit may include a 25-gauge needle or a 30-gauge needle. The product may include an inhaler device for aerosolizing/inhaling the composition comprising the ST6GAL-1. Thus, the ST6GAL-1 can be provided in a formulation suitable for aerosolization and oral inhalation by an inhaler device, or for example, a nebulizing device. Such products can therefore include STAGAL I provided in a liquid formulation in an ampule, such as a plastic, disposable ampule, containing an inhalation solution. In embodiments, separate doses can be provided in, for example, unit-dose low-density polyethylene (LDPE) vials, wherein each unit-dose LDPE vial is protected in a foil-pouch. The inhaler device can be any suitable instrument, such as those conventionally used to administer albuterol-sulfate containing compositions for treating asthmatic attacks, such as a metered-dose inhaler. In embodiments, the inhaler device can be configured to administer a powdered formulation, such as in the case of fluticasone propionate and salmeterol inhalation powder which is administered using a device sold under the trade name ADVAIR DISKUS. In embodiments, an portable injection device, similar to an auto-injector, i.e, a medical device for injecting a measured dose or doses of ST6GAL-1.

The printed material can be a label, or a paper insert, or can be printed on the packaging material itself. The printed material can provide information that identifies the ST6GAL-1 agent in the package, the amounts and types of other active and/or inactive ingredients, and instructions for taking or administering the composition, such as the number of doses to take or administer over a given period of time, and/or information directed to a pharmacist and/or another health care provider, such as a physician or nurse. The printed material can include an indication that the ST6GAL-1 pharmaceutical composition is for reducing inflammation or an allergic reaction in a subject. In an embodiment, the printed material can provide an indication that the composition is for reducing inflammation that is caused by an infectious agent or inflammatory dysregulation. In embodiments, the printed material can provide an indication that the composition is for reducing inflammation of an airway or an ear canal of the subject. In an embodiment, the infection is by Haemophilus influenza, such as nontypable H. influenza. In embodiments, the printed material can provide an indication that the composition is for reducing an allergic reaction, such as a type I hypersensitive reaction, such as an allergic reaction stimulated by a component of a particular food, or a component of an animal venom. In embodiments, the printed material can provide an indication that the composition comprising ST6GAL-1 is for oral inhalation for reducing airway inflammation. In embodiments the printed material can provide an indication that the composition comprising ST6GAL-1 is for intravenous administration for prophylaxis and/or therapy of sepsis, such as systemic inflammatory response syndrome, or SIRS, or septicemia.

In another aspect the disclosure includes a method for determining whether or not one or more test agents are suitable for use as anti-allergic agents. The method comprises exposing a ST6Gal-I deficient mouse to an allergic-reaction inducing agent, and introducing into the animal a test agent, wherein if the test agent prevents or reduces an allergic reaction in the mouse, the agent is a candidate for use in prophylaxis and/or therapy of allergic reactions. In embodiments, the allergic-inducing agent is a virus, such as adenovirus. In embodiments, the allergic reactions comprise acute allergic reactions, such as type 1 hypersensitivity reactions. In embodiments, the allergic reactions comprise anaphylaxis. For use in this method, suitable ST6Gal-I deficient mice are known in the art. In one embodiment, the ST6Gal-I deficient mouse comprises a specific disruption to the P1 promoter of the ST6Gal-1 gene, including removal of the 1.2-kb region containing Exon H. The St6gal1-dP1 mouse has a limited ST6Gal-1 deficiency restricted to the liver-produced pool of ST6Gal-1. The St6gal1-KO mouse has a globally inactivated ST6Gal-1 gene and was originally produced by Marth and co-workers (Martin L. T., Marth J. D., et al. (2002) Genetically altered mice with different sialyltransferase deficiencies show tissue-specific alterations in sialylation and sialic acid 9-O-acetylation. J. Biol. Chem. 277, 32930-32938). Such mice can be obtained, for example, from the Consortium of Functional Glycomics.

ST6Gal-1 (also referred to as ST6GalI and ST6GalI and ST6Gal-I) is a sialyltransferase that constructs the sialyl α2,6 to Gal β1,4GlcNAc glycan structure common on many cell surface and circulatory glycoproteins. Transcription of the ST6Gal-1 gene is mediated by 6 physically distinct promoter/transcriptional initiation regions. In the native form, ST6Gal-1 is localized in the Golgi, where it participates in the assembly of sialyl-glycoconjugates transiting the secretory apparatus. The intact catalytic domain can be proteolytically liberated and released into systemic circulation as the soluble ST6Gal-1 form. Therefore, ST6Gal-1 can be divided into two conceptual categories: The “cell-restricted” ST6Gal-1 that remains within the cells that produced them, and the “circulatory”, or “soluble”, ST6Gal-1 that has been released into systemic circulation. Circulatory ST6Gal-1 originates predominantly from the liver; specific inactivation of the liver-restricted promoter (P1) of the ST6Gal-1 gene results in depressed systemic ST6Gal-1 levels.

Liver synthesized ST6Gal-1 either remains in a cell-restricted manner and participates in sialylation of liver-derived circulatory glycoproteins, or it can be released into circulation as circulatory/systemic ST6Gal-1. Because inactivation of P1 results in negligible alteration to the sialylation of liver-derived serum glycoproteins, the principal and immediate biosynthetic consequence of P1 inactivation is the suppression of systemic ST6Gal-1 levels. However, there has been no previous recognition or demonstration that exogenously supplied ST6Gal-1 can affect hematopoiesis in vivo, especially for the purpose of providing a prophylactic and/or therapeutic effect.

For practicing the method of the invention, recombinant ST6Gal-1 can be isolated or synthesized using any suitable techniques, and commercially produced recombinant ST6Gal-1 is available from, for example, Novoprotein, Short Hills, N.J.

The amino acid sequence of human ST6Gal-1 proteins are known in the art. For reference, ST6Gal-1 amino acid sequences are as follows:

Complete sequence (as synthesized and as exist in the “cell-restricted” form as a 406aa protein (SEQ ID NO:1):

| | |
|---|---|
| 1 | mihtnlkkkf sccvlvfllf avicvwkekk kgsyydsfkl qtkefqvlks lgklamgsds |
| 61 | qsysssstqd phrgrqtlgs lrglakakpe asfqvwnkds ssknliprlq kiwknylsmn |
| 121 | kykvsykgpg pgikfsaeal rchlrdhvnv smvevtdfpf ntsewegylp kesirtkagp |
| 181 | wgrcavvssa gslkssqlgr eiddhdavlr fngaptanfq qdvgtkttir lmnsqlvtte |
| 241 | krflkdslyn egilivwdps vyhsdipkwy qnpdynffnn yktyrklhpn qpfyilkpqm |
| 301 | pwelwdilqe ispeeiqpnp pssgmlgiii mmticdqvdi yeflpskrkt dvcyyyqkff |
| 361 | dsactmgayh pllyeknlvk hlnqgtdedi yllgkatlpg frtihc |

Circulatory/systemic form 380aa protein (generated by proteolytic cleavage of parental “cell-restricted” form). Due to ambiguity of proteolytic action, the first 4-8 AA residues may or may not be present. Recombinant proteins used in the method of the invention may accordingly lack the first 4, 5, 6, 7 or 8 amino acids shown in the following sequence (SEQ ID NO:2)

kekk kgsyydsfkl qtkefqvlks lgklamgsds qsysssstqd
phrgrqtlgs lrglakakpe asfqvwnkds ssknliprlq
kiwknylsmn kykvsykgpg pgikfsaeal rchlrdhvnv
smvevtdfpf ntsewegylp kesirtkagp wgrcavvssa
gslkssqlgr eiddhdavlr fngaptanfq qdvgtkttir
lmnsqlvtte krflkdslyn egilivwdps vyhsdipkwy
qnpdynffnn yktyrklhpn qpfyilkpqm pwelwdilqe
ispeeiqpnp pssgmlgiii mmticdqvdi yeflpskrkt
dvcyyyqkff dsactmgayh pllyeknlvk hlnqgtdedi
yllgkatlpg frtihc

Additional information about the ST6Gal-1 sequence can be found in NCBI accession no. P15907 (Apr. 1, 1990 entry), which is incorporated herein by reference as it exists on the priority date for this disclosure.

The invention includes using recombinant ST6Gal-1 (also referred to as “rST6Gal-1” or “rST6G”) that is identical to the known human sequences shown above, or polypeptides that have an amino acid sequence that has greater than about 70% amino acid sequence identity, preferably about 75, 80, 85, 90, or 95% or more amino acid sequence identity, to the known sequence of human ST6Gal-1.

The rST6Gal-1 used in the invention may have conservative substitutions which are based generally on relative similarity of R— group substituents. As examples, these substitutions include gly or ser for als; lys for arg; gln or his for asn; glu for asp; ser for cys; asn for gln; asp for glu; ala for gly; asn or gln for his; leu or val for ile; ile or val for leu; arg for lys; leu or tyr for met; thr for ser; tyr for trp; phe for tyr; and ile or leu for val.

For use in the methods of the invention, a composition comprising rST6Gal-1 can be prepared as therapeutic formulations by mixing rST6Gal-1 with any suitable pharmaceutically acceptable carriers, excipients and/or stabilizers. Some examples of compositions suitable for mixing with the agent can be found in: Remington: The Science and Practice of Pharmacy (2005) 21st Edition, Philadelphia, Pa. Lippincott Williams & Wilkins. Thus, in various embodiments, the invention provides a pharmaceutical preparation comprising rST6Gal-1.

The compositions of the invention can be administered using any suitable method and route of administration. Some non-limiting examples include oral, parenteral, subcutaneous, intraperitoneal, intrapulmonary, and intranasal. Parenteral infusions include intramuscular, intravenous, intraarterial, intraperitoneal, pulmonary instillation as mist or nebulization, and subcutaneous administration.

Administration of the compositions of the invention can be performed in conjunction with conventional therapies that are intended to treat a disease or disorder described herein, wherein the conventional therapies entail or would benefit from modulation of hematopoietic homeostatic balance and/or controlling production of inflammatory cells, or their release into circulation and accumulation in inflammatory sites, or for reducing any other aspect of inflammation, or for reducing allergic reactions, including but not necessarily limited to type I hypersensitivity allergic reactions, and including anaphylaxis. Thus, in certain embodiments, compositions of this disclosure can be administered prior to, concurrently, or subsequent to administrations of anti-allergic medications, such as epinephrine, or synthetic epinephrine derivatives, such as salbutamol, or intravenous glucagon, or adjunct therapies, such as antihistamines, corticosteroids, or combinations of any of the foregoing. In embodiments, the disclosure includes pharmaceutical compositions comprising rST6Gal-1 and a distinct anti-allergic drug.

Routes and frequency of administration of the therapeutic compositions disclosed herein, as well as dosage, will vary from individual to individual, and may be readily established using standard techniques given the benefit of the present disclosure. Those skilled in the art will recognize how to formulate for pharmaceutical preparations comprising rST6Gal-1, and appropriate dosing can be determined by taking into account such factors as the size, age, gender and health of the individual to be treated, and the type and stage of disease or condition. The compositions comprising rST6Gal-1 can be administered prior to, concurrently, or subsequent to administration of other agents or the performance of any other medical protocol that is desirable for treating the individual.

In one embodiment, the individual to whom a composition comprising rST6Gal-1 is administered is at risk for or is experiencing a type I hypersensitivity allergic reaction. In embodiments, the individual is at risk for or is experiencing anaphylaxis from consuming a food-containing allergen, or from an envenomation. In embodiments, the composition comprising the rST6Gal-1 is administered such that the allergic reaction is inhibited. In embodiments, the allergic reaction is prevented.

The following examples are intended to illustrate, but not limit the invention.

### Example 1

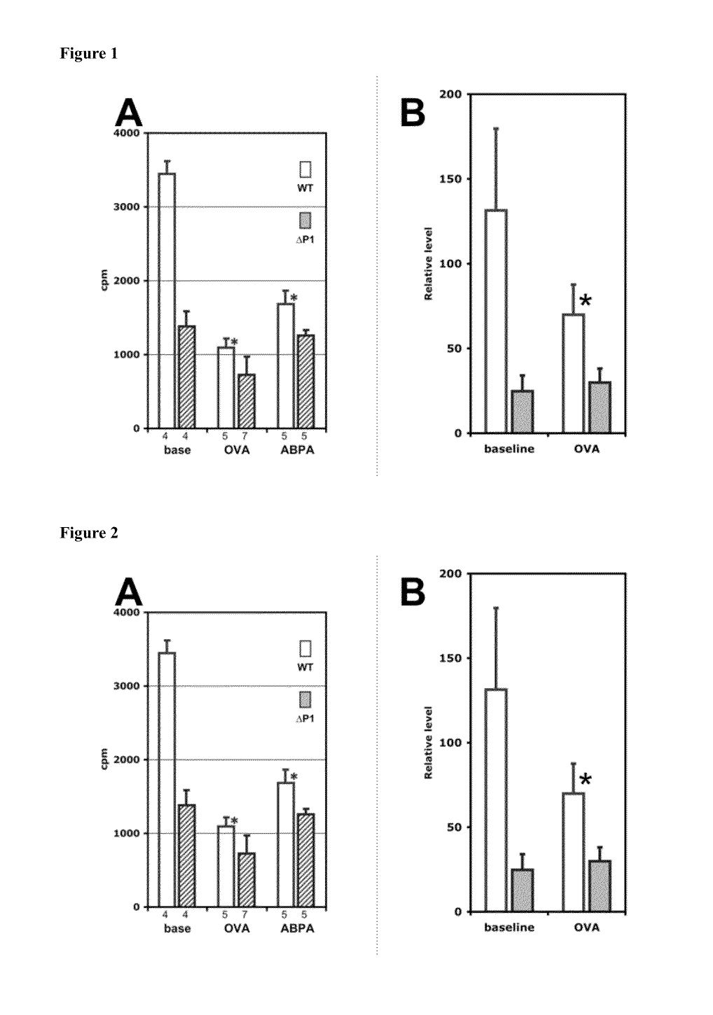

As will be evident from FIG. 1, this Example demonstrates that systemic ST6Gal-1 level is decreased during acute inflammation or during increased myelopoietic activity. In FIG. 1, panel A, serum sialyltransferase activity profiles are shown. Serum was harvested from wild-type (WT) and Siat1ΔP1 (ΔP1) mice either at rest (base) or undergoing OVA or ABPA protocols of allergic airway inflammation and tested for sialyltransferase activity. Shown is the 3 [H] incorporation into the synthetic acceptor substrate, GalNAc(β1,4)GlcNAc-o-Bz from 30 Ci/mmol CMP-3[H]NeuNAc by 10 μl serum after 2 hr incubation at 37° C. The numbers immediately beneath the abscissa indicate the number of animals comprising each data bar. Statistical significance is reached (p<0.01) for WT undergoing either OVA or ABPA when compared to baseline ( [symbol] ), and also for difference between WT and ΔP1 upon OVA provocation. Panel B, real time RT-PCR analysis of liver ST6Gal-1 mRNA if WT and ΔP1 mice either at rest (baseline) or undergoing the OVA protocol. Open bar is represents the wild-type (N=3) and hatched bar represents the Siat1ΔP1 (ΔP1) mice (N=3). Statistical significance was reached (p<0.05) for wild-type upon OVA provocation compared to baseline.

### Example 2

This Example demonstrates greater neutrophilia in Siat1ΔP1 and Siat1-null animals. As will be evident from FIG. 2, G-CSF mediated release of granulocytes from bone marrow is enhanced in systemic ST6Gal-1 deficient mice. To obtain the data summarized in FIG. 2, peripheral blood was collected in mice in the absence of treatment (resting, left panel) or 30 minutes after administration of G-CSF i.v. (right panel). The collected blood was analyzed by flow cytometry after lysis of the red blood cells. The granulocyte population, which are Gr-1 positive, was calculated by taking the percentage of the Gr-1 positive cells against total events for each flow acquisition. * marks mutant animal data point that was statistically different from wild-type animals by T test (p<0.05).

### Example 3

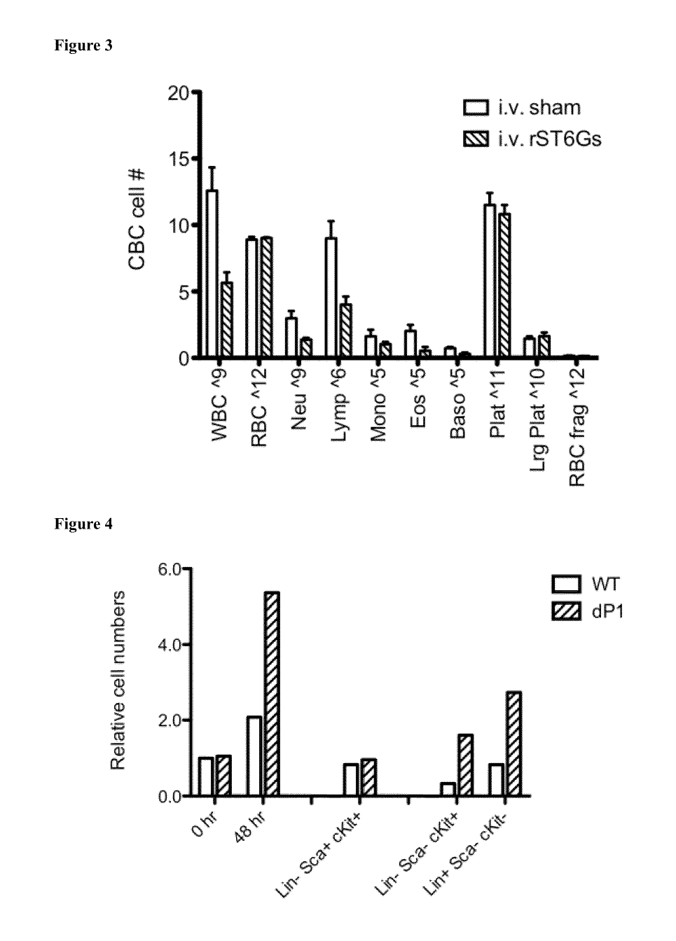

It will be evident from FIG. 3 that G-CSF mediated release of granulocytes is suppressed by i.v. infusion of recombinant ST6Gal-1. To obtain the data presented in FIG. 3, recombinant ST6Gal-1 or PBS vehicle (sham) was infused into recipient C57BL/6 wild-type mice. 24 hours after infusion, blood was withdrawn and subjected to CBC analysis. WBC: total white blood cells, RBC: red blood cells; Neu: neutrophil; Lymph: lymphocytes; Mono: monocytes; Eos: eosinophils; Baso: basophils; Plat: platelets. Differential counts between rST6G and sham injected groups were statistically significant in WBC, Neu, Lymph, and Eos categories.

### Example 4

FIG. 4 and FIG. 5 demonstrate that hematopoietic stem/progenitor cell proliferation and differentiation is elevated in systemic ST6Gal-1 deficient mice, but differentiation and proliferation is suppressed in the presence of recombinant ST6Gal-1. In particular, data summarized in FIG. 4 show that LSK (Lin−:Sca+:cKit+) cells from Siat1ΔP1 mice have greater proliferation in vitro. To obtain the data for FIG. 4, LSK cells isolated from C57BL/6 wild-type and from Siat1ΔP1 animals were cultured in vitro for 48 hours, and the total cells renumerated. The results showed that, starting from the same number of LSK cells of either genotype, Siat1ΔP1 proliferated 2.8-fold greater with greater proportion of cells differentiating into Sca-neg and Lin-pos populations after 48 hrs. Data summarized in FIG. 5 show that supplementation of in vitro cultures of C57BL/6 bone marrow cells with recombinant ST6Gal-1 (rST6G) resulted in depressed IL-3/G-CSF dependent and IL-5 dependent colonies. To obtain the data summarized in FIG. 5, bone marrow cells from wild-type animals were seeded into semi-solid media in the presence of either IL3 and G-CSF (left panel) or IL-5 (right panel), either with or without addition of rST6G. Seven days later, the cultures were assessed for total numbers (total) of colonies as well as the CFU type. 2- to 3-fold suppression of colony numbers by the presence of recombinant ST6Gal-1 was observed for G-, GM-, and Eos-CFU's.

### Example 5

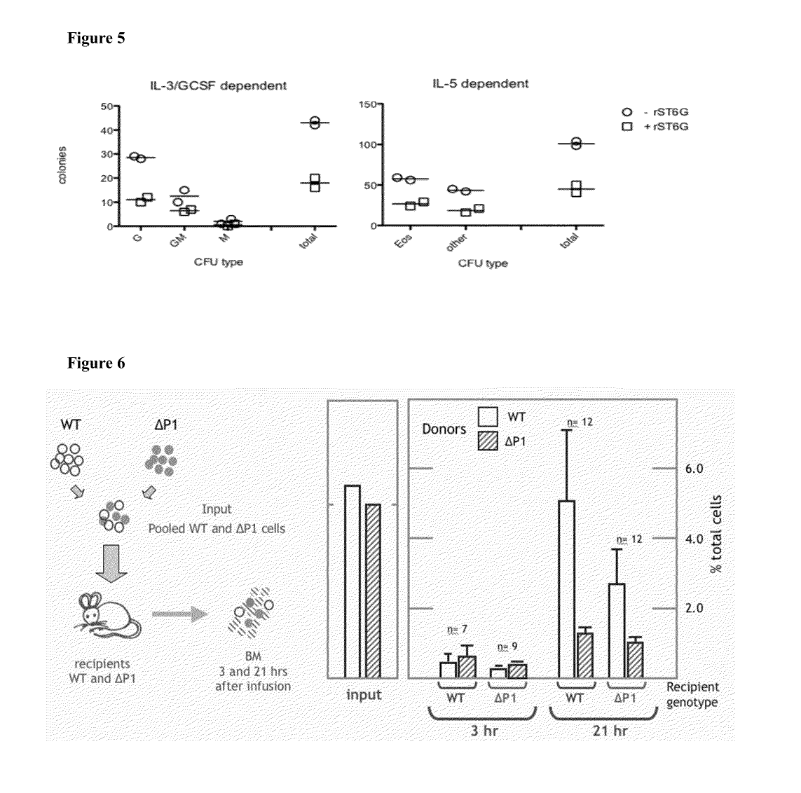

As will be evident from FIG. 6, ST6Gal-1 deficient animals are less able to retain transfused donor stem cells. In particular, C57BL/6 wild-type and Siat1ΔP1 donor cells were respectively labeled with different fluorescent dyes, mixed in 1:1 ratio and infused into either wild-type or Siat1ΔP1 recipients by tail vein injection. The bone marrow of the recipients were harvested 3 and 21 hrs after transfusion, and enumerated for the number of the respective donor cells. WT and Siat1ΔP1 cells homed with roughly equal efficiency, but the homing efficiency was significantly diminished in Siat1ΔP1 recipients (3 hr point). Stem cell retention was monitored in the 21 hr point, and significantly less Siat1ΔP1 cells were retained than wild-type cells. Moreover, the Siat1ΔP1 recipients had significantly less retained donor cells of either genotype than wild-type recipients.

### Example 6

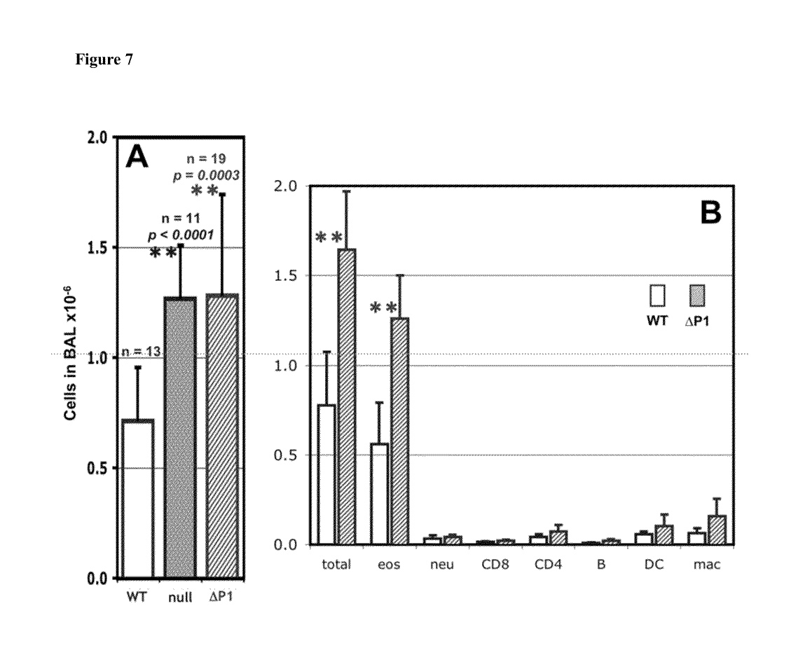

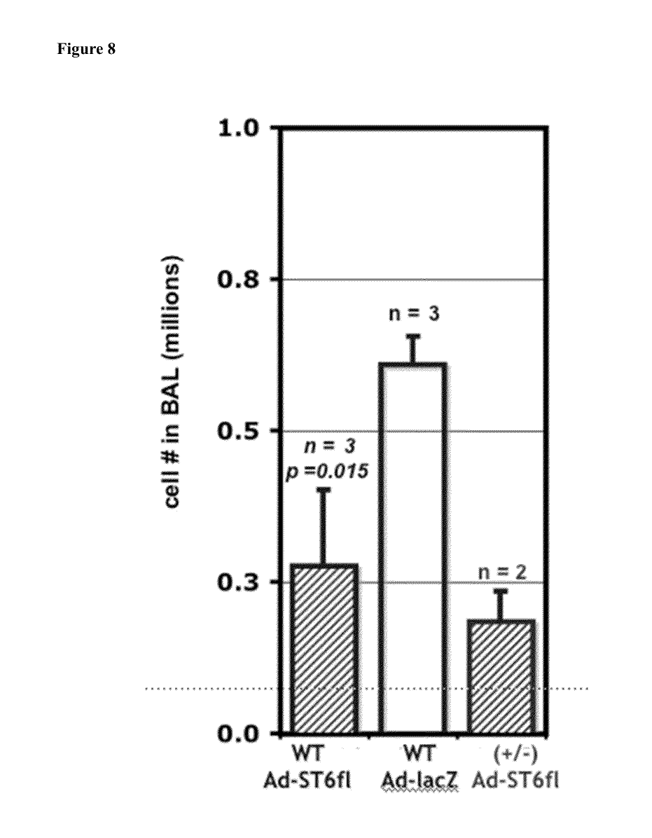

This Example shows that acute allergic airway inflammation is more severe in ST6Gal-1 deficient animals and that pulmonary inflammatory cell numbers are strikingly attenuated by increasing systemic ST6Gal-1 by adenoviral-mediated therapy. In particular, FIG. 7 shows elevated eosinophil infiltration in the bronchoalveolar lavage of ST6Gal-1-deficient mice upon allergen provocation. To obtain the data summarized in FIG. 7, bronchoalveolar lavage (BAL) was recovered from wild-type C57BL/6 (WT), Siat1ΔP1 (ΔP1), or Siat1-null (Null) animals undergoing acute OVA-provoked allergic pulmonary inflammation. Panel A shows the total cell content, expressed as BAL cells/animal, as determined on a coulter counter. N is the number of animals used for each respective data point. Panel B shows the BAL cell composition of WT (open bars) and Siat1ΔP1 (hatched bars) as determined by FACS analysis. The data shown is the mean of 4-5 WT and 4-6 Siat1ΔP1 animals, and [symbol] [symbol] denotes statistical significance of p<0.003). FIG. 8 demonstrates that allergic airway inflammation is attenuated by bolstering systemic ST6Gal-1. WT (+/+) or mice heterozygous for the Siat1-null mutation (+/−) were inoculated with either Ad-ST6fl or Ad-lacZ (i.v. 108 particles/animal) 5 days prior to OVA challenge. “n” is the number of mice for each respective data point, and “p” is the statistical difference between WT mice receiving Ad-lacZ or Ad-ST6fl.

### Example 7

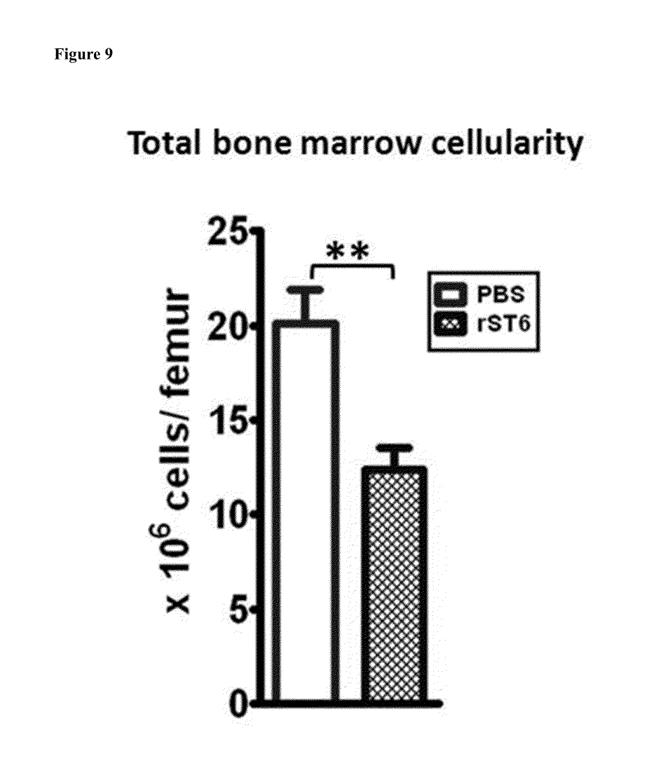

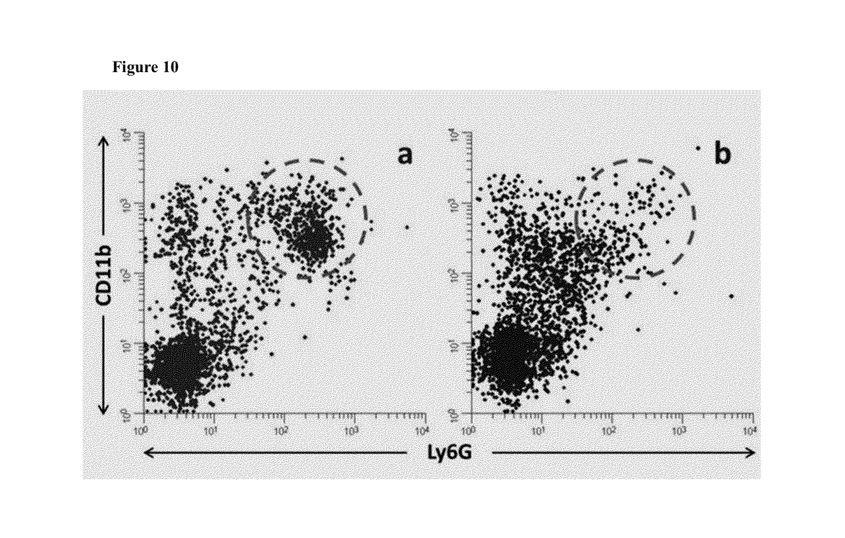

This Example demonstrates that administration of exogenous rST6G I effectuates a reduction in bone marrow cellularity as depicted in FIG. 9. Further, the data depicted in FIG. 10 (panels A, B) (flow analysis of bone marrow nucleated cells for CD11b and Ly6G) demonstrates depletion of granulocyte reservoir in marrow (circled population). Further still, FIG. 11 demonstrates that the administration of exogenous r ST6Gal-1 results in a decrease of total white cell counts (WBC), lymphocytes (LYMPH), and platelets (PLT) in circulation. To obtain the data presented in FIGS. 8, 9 and 10, animals (C57BL/6, males) received 200 ul rST6G (10 mg/Kg rST6G), or saline (PBS), by intravenous injections (3 sequential injections, 8 hours apart). Eight hours after the last injection, circulatory blood counts (CBC) were determined Nucleated bone marrow cells were extracted from the femurs and counted using TC10 Automated Cell Counter (Bio-Rad). Flow cytometry was performed using APC anti-mouse Ly6G antibody, FITC anti-mouse CD11b antibody and PE anti-mouse CD45R/B220 antibody. This experiment was performed with 6 PBS and 6 r ST6Gal-1 treated mice. The recombinant ST6Gal-1 (rST6G) was the recombinant human catalytic domain was captured as part of the Glycoenzyme repository in pDONR221

### Example 8

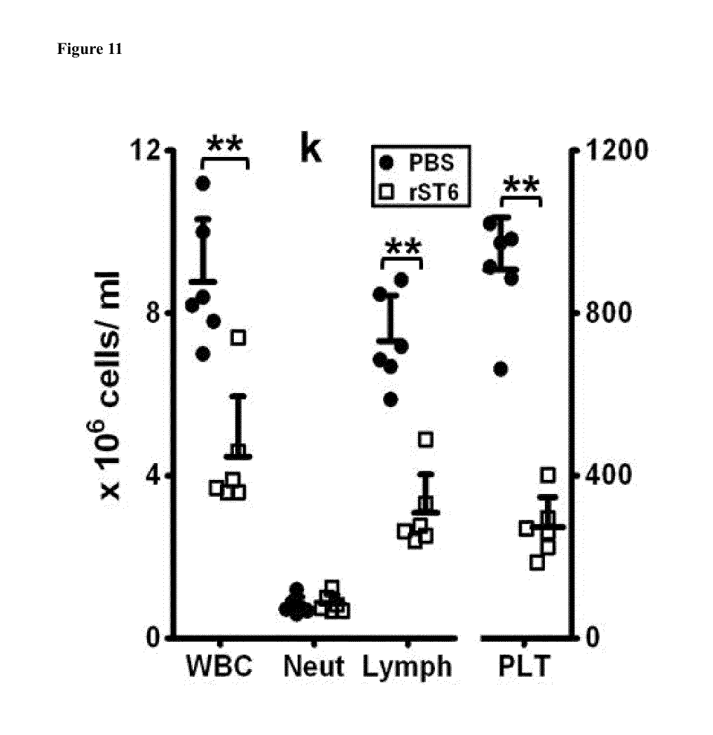

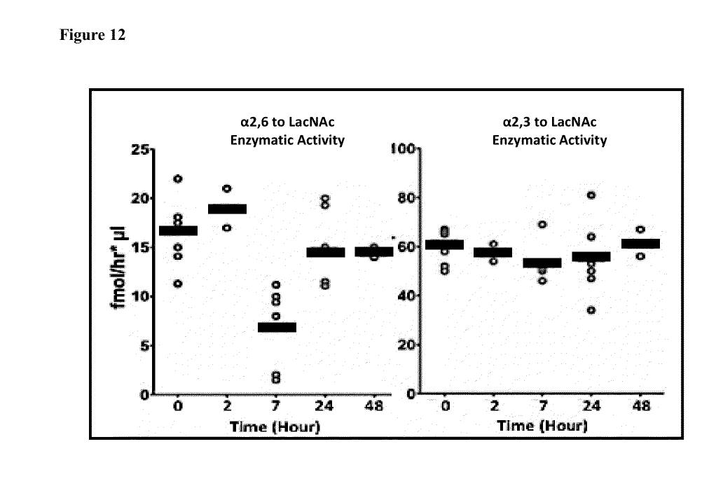

This Example demonstrates that transient depression of circulatory ST6Gal-1 accompanies acute airway inflammation. To obtain the data shown in FIG. 12, live NTHi bacterium (106 CFU/animal) was delivered by oropharengeal instillation, and blood was taken at the times shown after instillation. Sialyltransferase activities in the sera were measured by following transfer of CMP-[3H]Sia to Galβ1-4GlcNAc-O-Bn (LacNAc). The Siaα2,6 product, formed by ST6Gal-1 (Left Panel), was separated from Siaα2,3 product, form by various ST3Gal transferases (Right Panel), by SNA-agarose chromatography.

### Example 9

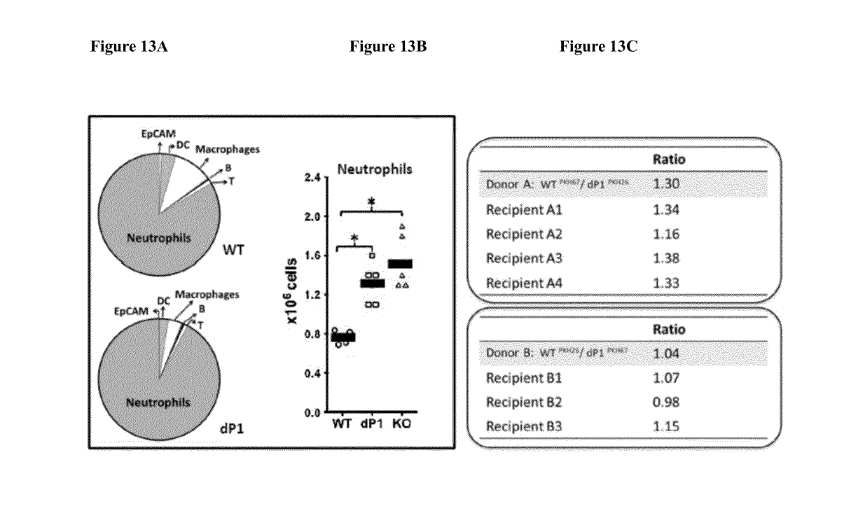

This example demonstrates greater neutrophilic acute airway in animals with depressed circulatory ST6Gal-1 levels. Wild type C57BL/6 (WT), ST6Gal1-dP1 (dP1), and ST6Gal1-KO (KO) mice were exposed to 10⁶ CFU of live NTHi bacterium by oropharengeal instillation. 24H after, the broncheo alveolar lavage fluid (BALF) was collected, counted, and leukocyte composition determined by flow cytometry. FIG. 13, Panel A, top shows the cellular composition of WT BALF, consisting predominantly of neutrophils (83.5%). Macrophage (10.5%), dendritic cells (DC, 3.8%), T (1%) and B (0.5%) cells, with a minor constituent of epithelial cells as defined by EpCAM (0.7%) made up the remainder of the BALF cells. Panel A, bottom, shows dP1 BALF composition, which was essentially identical to WT BALF in percentage contribution from the assessed cell types. FIG. 13, Panel B shows the total neutrophil numbers recovered from the BALF of NTHi-instilled animals, showing greater neutrophilic inflammation in dP1 and KO, compared to WT (1.6 and 2.0-fold, respectively). * denotes statistical significance with p<0.05 for the difference between value sets of dP1 or KO compared to WT. FIG. 13, Panel C shows that dP1 neutrophils are not preferentially recruited into the airway, compared to WT neutrophils. Neutrophils from the marrows of WT and dP1 mice were isolated by negative selection (see Material and Methods). The cells from each genotype were stained with one of the two distinct membrane dyes (red PKH-26 and green PKH-67), mixed in an approximately 1:1 ratio and transferred into recipient WT mice 2 hrs after NTHi challenge. The ratio of the dyed neutrophils recovered in the BALF 24 hrs after NTHi challenge are shown. Panel C, top, shows the donor mix of PKH67-labeled WT and PKH26-labeled dP1 neutrolphils yielding an initial ratio of 1.30, and the ratios of recovered labeled BALF neutrophils in 4 separate recipient animals. Panel C, bottom, shows the dye swap of PKH26-WT and PKH67-dP1 neutrophil donor ratio of 1.04 transfused into 3 separate recipient animals.

### Example 10

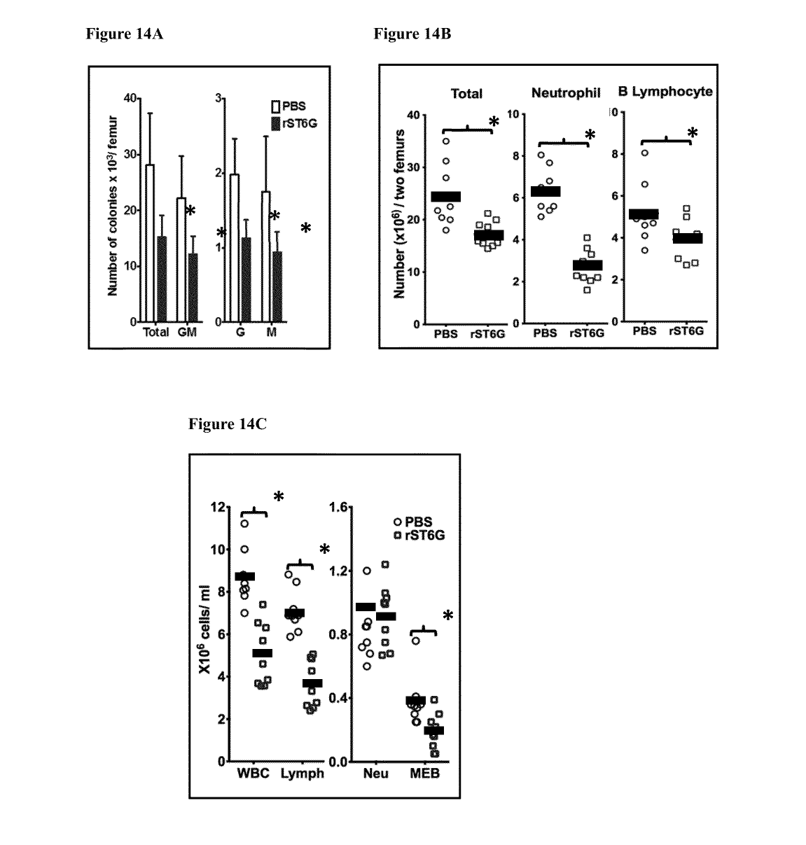

This Example demonstrates that intravenous rST6G administration depresses myelopoiesis and alters inflammatory cell availability. To obtain the data shown in FIG. 14, wild-type C587BL6 mice receiving either a single bolus of 150 ug of recombinant ST6Gal-1 (rST6G) or saline (PBS) were sacrificed 7 hours later. Bone marrow cells from hind limbs were isolated and analyzed as follows. FIG. 14, Panel A: marrow progenitor clonogenic activity was assessed as granulocyte/monocyte (GM), granulocyte (G), or monocyte (M) progenitor colonies. The combined total colony formed is also shown (Total). The results were from 9 saline- (open bars) and 9 rST6G-treated mice, where 4×104 marrow cells were plated in Methocult M3534 that promoted the growth of myeloid progenitors for 10 days. * denotes as p<0.01. FIG. 14, Panel B summarizes the overall bone marrow cellularity of PBS (round symbols) and rST6G-treated (square symbols) animals, where each symbol denotes each individual animal. Total bone marrow cellularity (Total), and neutrophil and B cell content are shown. * denotes p<0.01. FIG. 14, Panel C summarizes white cell counts in the blood as total white blood cell (WBC) count and differential count for lymphocyte (Lymph), neutrophil (Neu), monocyte, eosinophil and basophil (MEB). PBS (n=8) or rST6G-treated animals (n=9) were used. * denotes p<0.01

### Example 11

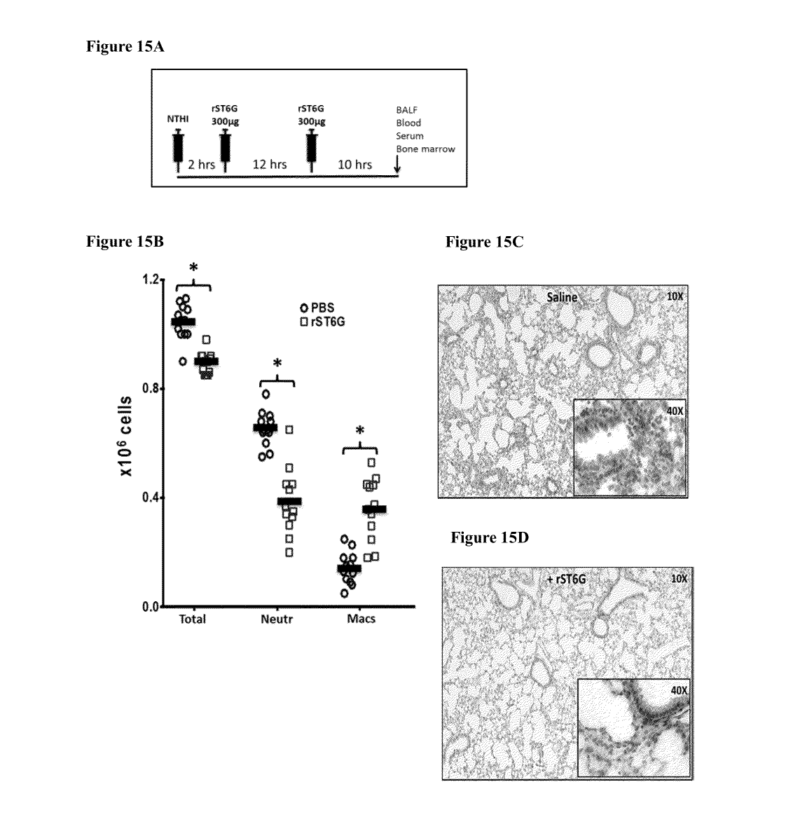

This Example demonstrates mitigation of neutrophilic infiltration in an acute airway by recombinant ST6Gal-1. FIG. 15, Panel A, top, is the schematic for the intervention protocol, where each animal received 2 injections of 300 ug of rST6G, or saline, spaced 12 hrs apart, and the first injection at 2 hrs after NTHi challenge. Animals were sacrificed at 24 hrs and assessed for pulmonary inflammation. FIG. 15, Panel A, bottom, shows the sustained elevation of circulatory ST6Gal-1 activity in animals that received rST6G. FIG. 15, Panel B: Inflammatory cell accumulation in the BALF of the rST6G- (rST6G) and shame- (PBS) treated animals showing total BALF cells (Total) and neutrophil accumulation (Neutr). * denotes p<0.01 FIG. 15, Panels C and D show the lung pathology of saline and rST6G-treated animals, respectively, having undergone the protocol outlined in Panel A. Blinded histopathologic evaluation showed consistent reductions in pulmonary inflammation among animals treated with rST6G. Compared to animals receiving saline, the rST6 treated group showed smaller inflammatory cuffs around bronchovascular bundles and fewer inflammatory cells within alveolar walls and alveolar spaces. The proportion of neutrophils within the infiltrate was also markedly reduced in the ST6G treated group.

### Example 12

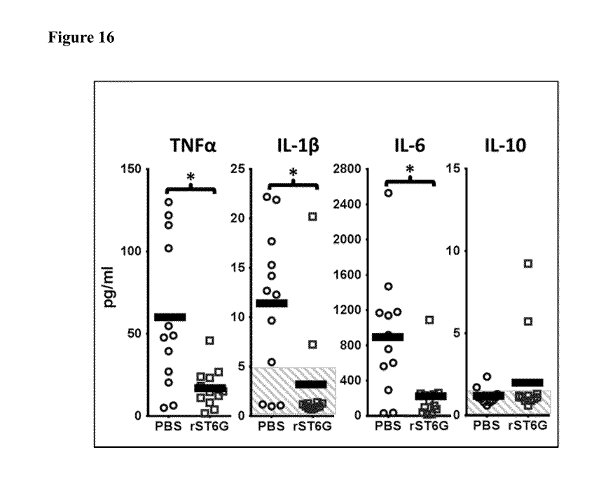

This Example demonstrates that rST6G suppresses inflammatory cytokine release during acute airway inflammation. C57BL/6 wild-type animals were challenged with NTHi and subjected to the rST6G or sham (PBS) treatment protocol as outlined in FIG. 15A. The bronchial alveolar lavage fluids (BALF) were analyzed for cytokines by Luminex 100 multiplex assays. Dashed lines and the shaded boxed regions shown in FIG. 16 represent the reliable lower assay limit of detection for each cytokine. Many of the obtained values, especially for the IL-10 assays and the Rst6G-treated cohorts for IL-1β values, were zero. For these, a default low value of “1” was assigned in order to calculate the p value. “*” denotes p<0.001.

### Example 13

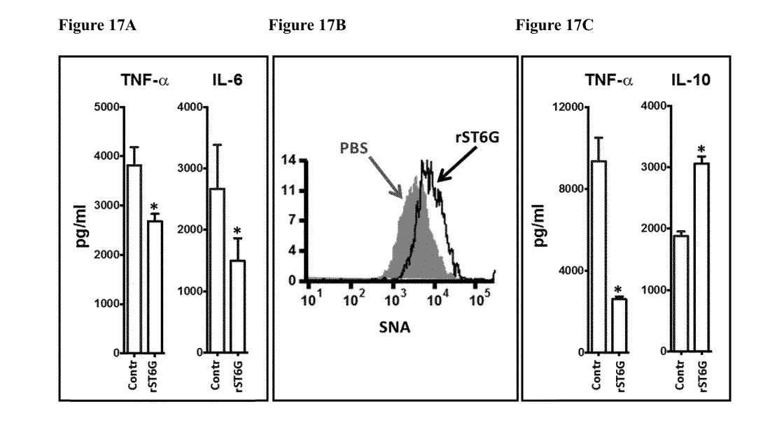

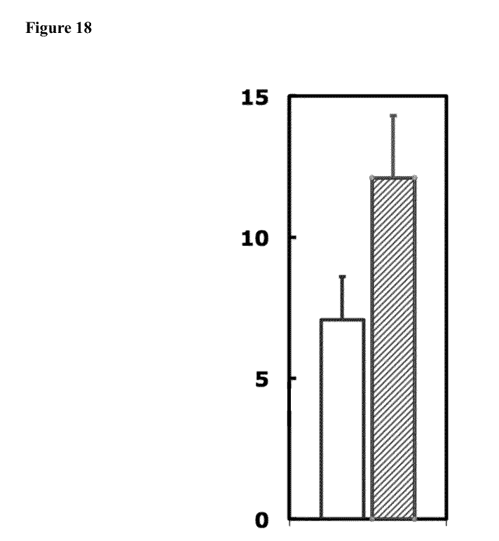

This Example demonstrates that inflammatory cytokine release by macrophage is attenuated by ST6Gal-1. The data are presented in FIG. 17. For FIG. 17, Panel A: Macrophage was recovered from the BALF of 3 wild-type C57BL/6 mice (at rest), and pooled. The pooled macrophage was divided into 5 separate but identical wells for each determination. Groups of 5 macrophage wells were exposed ex vivo to 100 ng/ml LPS either in absence or presence (20 ug/ml) of rST6G and CMP-Sia (100 uM) for 24 hrs, and the concentration of TNF-α and IL-6 released into the media were assessed by ELISA as described in Materials and Methods. “*” denotes p<0.001) For FIG. 17, Panel B: BALF macrophage was recovered from C57BL/6 animals 24 hrs after oropharyngeal challenge with NTHi, and assessed for cell surface reactivity to the α2,6-sialic acid-specific lectin, Sambucus nigra agglutinin (SNA) conjugated with FITC. The animals were either treated with rST6G (rST6G) or saline (PBS) under the protocol described in FIG. 15a. For FIG. 17, Panel C: Bone marrow-derived macrophage were generated from marrow cells of C57BL/6 animals. The identically seeded cells, in groups of 5 wells, were exposed to LPS in absence (control) or presence of rST6G (20 ug/ml) and CMP-Sia (100 uM) for 24 hrs, whereupon TNF-α and IL-10 released into the media were assessed by ELISA. Statistical significance was determined from by the concentration values taken from five separate wells for each condition. This data is representative of six separate experiments. “*” denotes p<0.001).

### Example 14

This Example demonstrates that the presence of ST6Gal-1 in an animal model during simulation of an acute allergic reaction can protect the animal from death. In this regard, we unexpectedly discovered that administration of adenovirus to STGal-1 deficient mice is lethal, whereas mice that express STCGal-1 survive the adenoviral challenge. Thus, based on the foregoing data presented herein, it is reasonable to expect that administration of exogenous STGal-1 will confer a prophylactic and/or therapeutic effect to an individual who is re-exposed to an allergen that would otherwise induce a Type I hypersensitivity (or immediate hypersensitivity) allergic reaction. The animal data are summarized in the following Table.

| | Starting | Survival at | Survival, |
|---|---|---|---|
| | numbers | 90 minutes | 24 hrs |
| C57BL/6, wild-type | 8 | 8 (100%) | 8 (100%) |
| St6gal1-dP1 | 7 | 0 (0%) | 0 (0%) |

St6gal1-dP1 animals are susceptible to sudden death upon intravenous inoculation with adenovirus. St6gal1-dP1 mice, with depressed circulatory ST6Gal-1, and wild-type C57BL/6 mice were inoculated intravenously with 10⁹ infectious units of adenoviral vector expressing LacZ in 100 μl saline. Lethality was100% among the St6gal1-dP1 receiving adenovirus but not saline alone (sham; not shown). All C57BL/6 animals survived the treatment.

While the invention has been described through illustrative examples, routine modifications will be apparent to those skilled in the art, which modifications are intended to be within the scope of the invention.

We claim:

1. A method for prophylaxis and/or therapy of inflammation or an allergic reaction in an individual in need thereof, the method comprising administering to the individual a composition comprising an effective amount of recombinant α2,6-sialyltransferase (rST6Gal-1), wherein the administration results in a reduction or inhibition of inflammation or an allergic reaction in the individual.

2. The method of claim 1, wherein the individual in need is experiencing an allergic reaction.

3. The method of claim 2, wherein the allergic reaction is a Type I hypersensitivity allergic reaction.

4. The method of claim 3, wherein the administering comprises an intravenous injection of the composition comprising the rST6Gal-1.

5. The method of claim 1, wherein the individual is in need of therapy for inflammation.

6. A method for prophylaxis and/or therapy of an autoimmune diseases in an individual in need thereof comprising administering to the a composition comprising an effective amount of rrST6Gal-1, wherein the administration results in a reduction of severity of the autoimmune disease.

7. The method of claim 6, wherein the autoimmune disease is selected from celiac disease, Crohn's disease, diabetes mellitus type 1, eosinophilic fasciitis, eosinophilic gastroenteritis, gastritis, Graves' disease, hypogammaglobulinemia, idiopathic inflammatory demyelinating diseases, thrombocytopenic purpura, rheumatoid arthritis, lupus erythematosus, myasthenia gravis, pernicious anaemia, psoriasis, Sjögren's syndrome, ulcerative colitis, and combinations thereof.

8. A method for identifying an agent as a candidate for use in treating an allergic condition, the method comprising exposing a ST6Gal-I deficient mouse to an allergic-reaction inducing agent, and introducing into the animal a test agent, wherein if the test agent prevents or reduces an allergic reaction in the mouse, the agent is a candidate for use in prophylaxis and/or therapy of the allergic condition.

9. The method of claim 8, wherein the ST6Gal-I deficient mouse comprises a disruption of the P1 promoter of the ST6Gal-1 gene.

10. The method of claim 8, wherein the allergic-reaction inducing agent comprises an adenovirus.


SEQUENCE LISTING (2 sequences)

SEQ ID NO: 1 - protein, 406 aa, human
MIHTNLKKKFSCCVLVFLLFAVICVWKEKKKGSYYDSFKLQTKEFQVLKSLGKLAMGSDSQSVSSSSTQDPHRGRQTLGSLRGLAKAKPEASFQVWNKDSSSKNLIPRLQKIWKNYLSMNKYKVSYKGPGPGIKFSAEALRCHLRDHVNVSMVEVTDFPFNTSEWEGYLPKESIRTKAGPWGRCAVVSSAGSLKSSQLGREIDDHDAVLRFNGAPTANFQQDVGTKTTIRLMNSQLVTTEKRFLKDSLYNEGILIVWDPSVYHSDIPKWYQNPDYNFFNNYKTYRKLHPNQPFYILKPQMPWELWDILQEISPEEIQPNPPSSGMLGIIIMMTLCDQVDIYEFLPSKRKTDVCYYYQKFFDSACTMGAYHPLLYEKNLVKHLNQGTDEDIYLLGKATLPGFRTIHC

SEQ ID NO: 2 - protein, 380 aa, human
KEKKKGSYYDSFKLQTKEFQVLKSLGKLAMGSDSQSVSSSSTQDPHRGRQTLGSLRGLAKAKPEASFQVWNKDSSSKNLIPRLQKIWKNYLSMNKYKVSYKGPGPGIKFSAEALRCHLRDHVNVSMVEVTDFPFNTSEWEGYLPKESIRTKAGPWGRCAVVSSAGSLKSSQLGREIDDHDAVLRFNGAPTANFQQDVGTKTTIRLMNSQLVTTEKRFLKDSLYNEGILIVWDPSVYHSDIPKWYQNPDYNFFNNYKTYRKLHPNQPFYILKPQMPWELWDILQEISPEEIQPNPPSSGMLGIIIMMTLCDQVDIYEFLPSKRKTDVCYYYQKFFDSACTMGAYHPLLYEKNLVKHLNQGTDEDIYLLGKATLPGFRTIHC